(19) United States
(12) Patent Application Publication
(10) Pub. No.: US 2015/0239814 A1
(43) Pub. Date: Aug. 27, 2015

(54) PROCESS FOR PHOTOCATALYTIC ACCEPTOR-FREE DEHYDROGENATION OF ALKANES AND ALCOHOLS

(71) Applicants: Robert FRANKE, Marl (DE); Jennifer Julis, Duesseldorf (DE); Dirk Fridag, Haltern am See (DE); Katrin Marie Dyballa, Recklinghausen (DE); Nico Weding, Luedenscheid (DE); Abhishek Dutta Chowdhury, West Bengal (IN); Matthias Beller, Nienhagen (DE); Ralf Jackstell, Cuxhaven Altenwalde (DE); Ivana Fleischer, Regensburg (DE); Beatrice Percia, Vadamadurai (IN)

(72) Inventors: Robert FRANKE, Marl (DE); Jennifer Julis, Duesseldorf (DE); Dirk Fridag, Haltern am See (DE); Katrin Marie Dyballa, Recklinghausen (DE); Nico Weding, Luedenscheid (DE); Abhishek Dutta Chowdhury, West Bengal (IN); Matthias Beller, Nienhagen (DE); Ralf Jackstell, Cuxhaven Altenwalde (DE); Ivana Fleischer, Regensburg (DE); Beatrice Percia, Vadamadurai (IN)

(73) Assignee: Evonik Industries AG, Essen (DE)

(21) Appl. No.: 14/630,018

(22) Filed: Feb. 24, 2015

(30) Foreign Application Priority Data
Feb. 25, 2014 (DE) 102014203341.1

(51) Int. Cl. C07C 45/00; C07C 5/333

(52) U.S. Cl. CPC C07C 45/002; C07C 5/333; C07C 2531/20; C07C 2531/02

(57) ABSTRACT

A process for photocatalytic acceptor-free dehydrogenation of alkanes and alcohols, in which an alkane or an alcohol is irradiated in the presence of a rhodium complex containing organic phosphorus(III) compounds as ligands as a catalyst, and in the presence of at least one Lewis base is provided.

### CROSS REFERENCE TO RELATED APPLICATIONS

This application claims priority to German Application No. 102014203341.1, filed Feb. 25, 2014, the disclosure of which is incorporated herein by reference in its entirety.

### BACKGROUND

The invention relates to a process for photocatalytic acceptor-free dehydrogenation of alkanes and alcohols.

Alkenes may be regarded as the most important and versatile raw materials in the chemical industry. In contrast to the alkanes, however, they are much less widely available. Therefore, the direct catalytic dehydrogenation of alkanes to alkenes has experienced a great deal of attention as one of the most efficient and economic routes to alkene formation in research activities [Choi, J., Roy MacArthur, A. H.; Brookhart, M.; Goldman, A. S. Chem. Rev. 2011, 111, 1761-1779].

Acceptor-free alkane dehydrogenation may be regarded as an ideal process because of its simplicity and atom economy. Such an atom-economic and acceptor-free dehydrogenation can be achieved under photocatalytic conditions. However, this reaction is limited by long reaction times, catalyst deactivation and lower reactivity compared to catalytic dehydrogenation/transfer hydrogenation reaction. Nomura et al. (J. Chem. Soc., Chem. Commun. 1988, 161-162) describe that a Rh(PMe₃)₂(CO)Cl catalyst is active both in acceptor-free photocatalytic dehydrogenation and in thermochemical transfer hydrogenation, but only under hydrogen pressure, which restricts the use thereof for synthesis of alkenes.

In order to achieve alkane dehydrogenation at relatively low temperature under homogeneous conditions, sacrificial olefins (acceptors) are normally used. In general, an efficient alkane transfer hydrogenation is conventionally achieved only with a large excess of a sacrificial olefin (up to a 20-fold excess), which restricts potential viable applicability. The turnover numbers (TON) in these methods, which are of industrial relevance, are also normally limited at about 1000 with sensible reaction times. Turnover numbers (TON)>1000 are only achieved with long reaction times of several days. Even though there have continuously been various efforts to achieve homogeneous catalytic alkane dehydrogenations in the last 30 years, there is considerable need for significant improvements, specifically for acceptor-free atom-economic alkane dehydrogenation.

At present, successful alkane dehydrogenations depend mainly on the behaviour of various specific demanding pincer ligands and the thermal stability thereof (including that of the metal complexes thereof). In order to overcome the high endothermicity of the alkane dehydrogenation, higher reaction temperatures of up to 250° C. or long reaction times of up to 3 days are normally employed. Therefore, reactivity is determined strictly by the thermal stability of the catalyst. A further problem is that of inhibition of the reaction by the olefin which is present either as the sacrificial olefin or as the product, especially in the case of relatively long reaction times.

It was therefore an object of the invention to develop a process for acceptor-free alkane dehydrogenation which avoids olefins as acceptors and permits an atom-economic alkane dehydrogenation, the intention being to avoid long reaction times, catalyst deactivation and low reactivity.

### SUMMARY OF THE INVENTION

This and other objects have been achieved according to the present invention, the first embodiment of which includes a process for dehydrogenation of an alkane or an alcohol, comprising: irradiating the alkane or alcohol in a reaction mixture comprising a rhodium complex and a Lewis base to remove hydrogen from the alkane or alcohol; wherein a hydrogen acceptor is not present, and the rhodium complex comprises a ligand of an organic phosphorus(III) compound.

In one variant of the first embodiment the dehydrogenation is conducted in a glass reactor or a metal reactor comprising glass.

In a further variant the hydrogen is removed from the reaction mixture.

In another variant, the rhodium complex is of formula (I):

Rh(L¹)₂(CO)X (I)

wherein L¹ is P(C₁-C₅alkyl)₃, and X is chloride, bromide or acetate.

In a further variant, the rhodium complex is of formula (II):

Rh₂(L²)₂(CO)₂(X)₂ (II)

wherein L² is P(Ph)₂(CH₂)ₙP(Ph)₂, n is from 1 to 3, and X is chloride, bromide or acetate.

The forgoing description is intended to provide a general introduction and summary of the present invention and is not intended to be limiting in its disclosure unless otherwise explicitly stated. The presently preferred embodiments, together with further advantages, will be best understood by reference to the following detailed description

### DETAILED DESCRIPTION OF THE PREFERRED EMBODIMENTS

As used herein, the words “a” and “an” and the like carry the meaning of “one or more.” The phrases “selected from the group consisting of,” “chosen from,” and the like include mixtures of the specified materials. Terms such as “contain(s)” and the like are open terms meaning ‘including at least’ unless otherwise specifically noted. Where a numerical limit or range is stated, the endpoints are included. Also, all values and subranges within a numerical limit or range are specifically included as if explicitly written out.

This invention is based on the surprising finding that a photocatalytic acceptor-free dehydrogenation of alkanes in the presence of at least one Lewis base enables an efficient atom-economic photocatalytic process by which alkanes and also alcohols may be dehydrogenated with high turnover numbers over a wide substrate range.

In contrast to the thermal dehydrogenation [Balsells, R. E.; Frasca, A. R. Tetrahedron 1982, 38, 245-255] of alcohols, the inventors are not aware of any report of studies of photocatalytic dehydrogenations of alcohols.

In one variant of the process, the process is used for photocatalytic acceptor-free dehydrogenation of alcohols.

In another variant of the process, the process is used for photocatalytic acceptor-free dehydrogenation of alkanes.

Thus in the first embodiment the present invention provides a process for dehydrogenation of an alkane or an alcohol, comprising:

irradiating the alkane or alcohol in a reaction mixture comprising a rhodium complex and a Lewis base to remove hydrogen from the alkane or alcohol; wherein

a hydrogen acceptor is not present, and the rhodium complex comprises a ligand of an organic phosphorus(III) compound.

In a variant of the process, the alkane or the alcohol has a carbon chain length of 2 to 30 carbon atoms. Preferably, the alkane or the alcohol has a carbon chain length of 2 to 12 carbon atoms, more preferably a carbon chain length of 4 to 10 carbon atoms.

Alkanes and alcohols preferably have a carbon chain length of 2 to 30 carbon atoms; iso forms and cyclic compounds are included. The compounds may be substituted. Specific examples of alkanes, cycloalkanes, alcohols and cycloalcohols to be used are given hereinafter.

Alkanes of the general formula CₙH₂ₙ₊₂ (n=2 to 30) used in the process according to the invention may, for example, be ethane, propanes, butanes, pentanes, hexanes, heptanes, octanes, nonanes, decanes, undecanes, dodecanes and the like. Cycloalkanes, which can be represented by the general formula CₙH₂ₙ (n=4 to 30) are, for example, cyclopentane, cyclohexane, cycloheptane, cyclooctane, cyclododecane, and alkyl-substituted alkanes, for example methylcyclohexane and the like.

The alcohols and cycloalcohols derive from the alkanes and cycloalkanes. Essentially, cycloalcohols may be more reactive substrates compared to linear or substituted acyclic alcohols under similar process conditions. Preference may be given to linear and branched aliphatic alcohols of the general formula CₙH₂ₙ₊₁OH (n=2 to 12) and cycloaliphatic alcohols of the general formula CₙH₂ₙ₋₁OH (n=5 to 12). Examples include: propanols, butanols, pentanols, hexanols, cyclohexanol, methylcyclohexanols, heptanols, octanols, cyclooctanol, nonanols, decanols, undecanols and dodecanols. In the process according to the invention, it is also possible to use substituted alkanes/cycloalkanes and substituted alcohols/cycloalcohols. Each substituent may be at least one alkyl group or aromatic group which may have a carboxyl group, ester group, halogen group, nitro group or methoxy group. But a carboxyl group, ester group, halogen group, nitro group or methoxy group are also possible substituents. Alkyl substituents preferably have 1 to 6 carbon atoms.

Preferred examples of the alkanes include n-butane, n-pentane, n-hexane, 2-methylpentane, n-heptane, n-octane, n-nonane, n-decane, n-dodecane. Preferred examples of cycloalkanes are methylcyclohexane, adamantane, cis-decalin, trans-decalin, cyclohexane and the like. The cycloalkanes include a condensed ring, and it is also possible for an aromatic ring and a cycloalkane ring to be fused in ortho positions. Examples thereof are indane, tetralin, fluorene and the like. Preferred alcohols/cycloalcohols are, for example, n-propanol, i-propanol, n-butanol, i-butanol, n-pentanol, n-hexanol, cyclohexanol, n-heptanol, n-octanol, cyclooctanol, n-nonanol and the like.

In another variant of the process, the Lewis base is an organic amine. In another variant of the process, the Lewis base may be a heterocyclic amine. In another variant of the process, the Lewis base may be a bipyridine. In another variant of the process, the Lewis base used may be 2,2-bipyridine or 4,4-bipyridine. In another variant of the process, the Lewis base used may be bathocuproin or phenanthroline.

In one variant of the process, the reaction may be conducted in the presence of CO₂.

It has additionally been found that, in the presence of CO₂, the reactions for dehydrogenation proceed better with higher yields in a longer reaction regime.

In one variant of the process, irradiation is effected with a light source which emits light with a wavelength range from 320 nm to 500 nm. The influence of the wavelength of the light source used has also been studied, and it was found that irradiation may preferably be effected with light in the wavelength range from λ=320 nm to 500 nm.

In one variant of the process, dehydrogenation may be conducted at a temperature of 45° C. to 120° C. The inventive dehydrogenation also takes place, in a particularly optimal manner, at a temperature of preferably 45° C. to 120° C. A temperature of 80° C. to 95° C. is particularly preferred. The best yields may be achieved at a reaction temperature of 85° C. to 90° C.

Furthermore, the use of a glass reactor or of a metal reactor including glass in the process according to the invention is particularly effective. Single-wall glass reactors, preferably having a wall thickness of 1.0 to 3.0 mm, are particularly suitable. Interestingly, barely any reaction takes place if merely a suitable metal reactor with a light inlet is used. Surprisingly, the presence of glass has a remarkable influence on the inventive reaction, and not only the material but also the wall thickness of the glass vessel used can be important. Thus, the reaction may be conducted with glass vessels of different wall thickness; a wall thickness of 1.2 mm to 1.8 mm has been found to be particularly optimal.

In one variant of the process, the glass pane has a thickness of 1.2 mm to 3.0 mm.

Advantageously, therefore, thin-wall glass vessels are used in the process according to the invention, since transmission therein is considerably greater and therefore more energy is available for alkane dehydrogenation. The inventors are not aware that this significant influence of the presence of glass and of the wall thickness of the reaction vessels in photocatalytic reactions has been previously reported.

In one variant of the process, single-wall glass reactors are used.

In one variant of the process, a rhodium complex of the general formula (I) is used:

Rh(L¹)₂(CO)X (I)

wherein L¹ is P(C₁-C₅alkyl)₃ and X is an anion selected from chloride, bromide and acetate.

In one variant of the process, L¹ in (I) is PMe₃ or PtBu₃.

In one variant of the process, X in (I) is chloride.

In one variant of the process, a rhodium complex of the general formula (II) is used:

Rh₂(L²)₂(CO)₂(X)₂ (II)

wherein L²=P(Ph)₂(CH₂)ₙP(Ph)₂ with n=1 to 3, and X is an anion selected from chloride, bromide, acetate.

In one variant of the process, L² in (II) is P(Ph)₂(CH₂) P(Ph)₂.

In one variant of the process, in (II), X is chloride.

In one variant of the process, the hydrogen formed is removed from the reaction mixture.

The present process permits atom-economic alkane dehydrogenation with short reaction times, wherein the catalyst remains stable. For example, with n-octane as model substrate in the absence of additives, a yield of only 3.5% of octenes, i.e. a TON of 333 in 3 h or a turnover frequency of 111 h⁻¹, was achieved.

In the presence of the Lewis base 4,4′-bipyridine, in contrast, in a glass vessel having a wall thickness of 1.2 mm, about 20% yield was achieved in the process according to the invention with a TON of 1518 and a TOF of 217 h⁻¹ within 7 hours. In the case of use of other substrates such as cyclooctane, about 40% of cis-cyclooctene may be achieved with a TON=2883 within 7 h. Analogously, for example, with methylcyclohexane, 16.5% yield may be achieved with a TON of 1200.

In order to conduct an even more effective reaction, the hydrogen formed may advantageously be removed from the reaction solution. This is effected, for example, by employing a high stirrer speed and constant gas flow (e.g. argon). This reaction regime may further increase the TONs achieved, and they are thus significantly higher than systems described in the literature.

The irradiation may preferably be effected with light, preferably over a wavelength range of λ=320 nm to 500 nm. The dehydrogenation may also be effected, in a particularly optimal manner, at a temperature of preferably 45° C. to 120° C., preferably at a temperature of 80° C. to 95° C. More particularly, a reaction temperature of 85° C. to 90° C. may be effective. In addition, the use of a glass reactor or of a metal reactor including glass in the process according to the invention may be particularly effective. Single-wall glass reactors, preferably having a glass thickness of 1.0 to 3.0 mm, may be particularly suitable. The rhodium complex used may preferably be one of the general formula (I), specified above, especially with L¹=PMe₃ or PBu₃ and X=Cl. In general, the preferred catalyst may be Rh(PMe₃)₂(CO)Cl.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

The invention is illustrated in detail hereinafter by working examples.

### EXAMPLES

General Reaction Conditions:

All synthetic operations were conducted under argon in dried Duran borosilicate glass vessels with suitable Schlenk techniques. The Rh(PMe₃)₂(CO)Cl catalyst was prepared analogously to a literature method [Bridgewater, J. S.; Netzel, T. L.; Schoonover, J. R.; Massick, S. M.; Ford, P. C. Inorg. Chem. 2001, 40, 1466-1476]. The products were analysed against a comparative sample and the yield by gas chromatography (Agilent 6890N network GC System with a (60 m×250 μm×0.25 μm) DB Wax column and isooctane as internal standard (0.2 ml, 1.2 mmol) after dilution with acetone. The response factors of each product were determined by means of a Multiple Point Internal Standard GC Quantitation Method′ against isooctane. For all analyses, the following conditions were chosen: N₂ as carrier gas, inlet temperature: 250° C., inlet pressure: 104.4 kPa, injection volume: 1.0 μl, split ratio: 100:1, split flow: 80.0 ml/min, flow rate: 0.8 ml/min until 20 min, which was then increased to 2.8 ml/min at 1.0 ml/min², temperature: 35° C. to 20 min, then increased at 40° C./min to 200° C. and then held at 200° C. for 15 min. Detector temperature: 250° C., hydrogen flow rate: 30 ml/min, air: 300 ml/min, nitrogen flow rate: 25 ml/min.

General Procedure 1:

The appropriate glass vessel, provided with a reflux condenser and magnetic stirrer, was charged under argon with 0.004 mmol of the Rh(PMe₃)₂(CO)Cl catalyst and 0.02 mmol of the appropriate additive. Very careful working under argon as protective gas was necessary, since the catalyst is deactivated very readily in the presence of atmospheric oxygen and light. Subsequently, 30 mmol of substrate were added and an argon stream was applied. The stirrer speed was set to 1000 min⁻¹ and the glass vessel was covered with aluminium foil. Then the Lumatec Superlite 400 light source used, which emits light over a wavelength range from 320 nm to 500 nm, was switched on. After the reaction, the light source was switched off, the reaction solution was cooled down and the yield was determined by gas chromatography with isooctane as internal standard. The turnover numbers (TON) in the tables were calculated as [mmol of product]/[mmol of catalyst].

General Procedure 2:

The appropriate glass vessel, provided with a reflux condenser and magnetic stirrer, was charged under argon with 0.004 mmol of the Rh(PMe₃)₂(CO)Cl catalyst and 0.02 mmol of the appropriate additive. Very careful working under argon as protective gas was necessary, since the catalyst is deactivated very readily in the presence of atmospheric oxygen and light. Subsequently, 30 mmol of substrate were added and an argon stream was applied. The stirrer speed was set to 1000 min⁻¹ and the glass vessel was covered with aluminium foil. A metal capillary was used to pass a CO₂ stream through the solution. Then the Lumatec Superlite 400 light source used was switched on. After the reaction, the light source was switched off, the CO₂ stream was stopped, the reaction solution was cooled down and the yield was determined by gas chromatography with isooctane as internal standard. The turnover numbers (TON) in the tables were calculated as [mmol of product]/[mmol of catalyst].

### Example 1

### Conversion of Additives Used in Accordance with the Invention for Dehydrogenation of n-Octane

$$\begin{matrix}n \text{-} octane \\ 30 mmol\end{matrix} {{\to }^{\begin{matrix}RhCl ( CO ) {( {PMe}_{3} )}_{2} ( 0.004 mmol ) + \\ additive ( 0.02 mmol )\end{matrix}}}_{ {h}^{*} v ( I = 320 - 500 nm ) , T = 85 - {88}^{°} C . } octenes$$

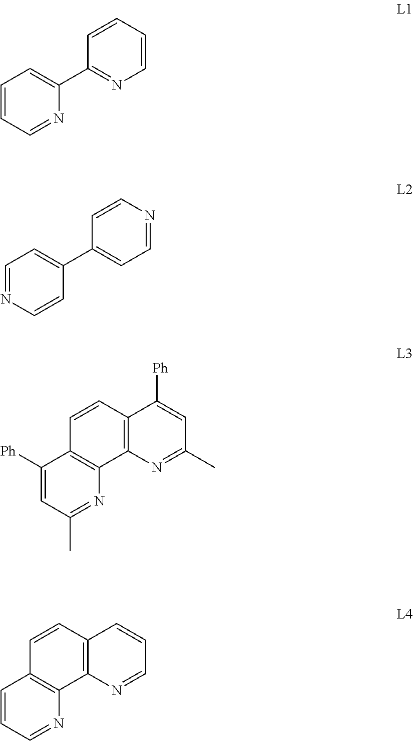 [SMILES: c1ccc(-c2ccccn2)nc1 ; c1cc(-c2ccncc2)ccn1 ; Cc1cc(-c2ccccc2)c2ccc3c(-c4ccccc4)cc(C)nc3c2n1 ; c1cnc2c(c1)ccc1cccnc12]

### Example 1.1

A jacketed three-neck photoreactor having a wall thickness of 2.3 mm, provided with a reflux condenser and magnetic stirrer, was charged with 1.3 mg of Rh(PMe₃)₂(CO)Cl (0.004 mmol) and 3.1 mg of 2,2′-bipyridine (L1, 0.02 mmol). Subsequently, the reaction vessel was evacuated and filled with argon three times, in order to achieve inert conditions. Then 5 ml of n-octane (30 mmol) were added. An argon stream was applied in order to remove hydrogen formed. The reactor was covered with aluminium foil and the light source having a wavelength of 320 nm to 500 nm (Lumatec Superlite 400) was switched on. The mixture was stirred at 1000 min⁻¹ for three hours. After the reaction, the light source was switched off and the reaction solution was cooled down. The yield was determined by gas chromatography with isooctane as internal standard.

### Example 1.2

In analogy to Example 1.1, 3.1 mg of 4,4′-bipyridine (L2, 0.02 mmol) were used as additive.

### Example 1.3

In analogy to Example 1.1, 3.1 mg of 4,4′-bipyridine (L2, 0.02 mmol) were used as additive and the mixture was stirred at 1000 min⁻¹ for five hours.

### Example 1.4

In analogy to Example 1.1, 7.2 mg of bathocuproin (L3, 0.02 mmol) were used as additive.

### Example 1.5

In analogy to Example 1.1, 3.6 mg of phenanthroline (L4, 0.02 mmol) were used as additive. Yield and conversion rates are shown in Table 1.

TABLE 1

| No. | Additive | Time (h) | Yield (%) | TON | TOF (h⁻¹) |
|---|---|---|---|---|---|
| 1.1 | 2,2′-bipyridine (L1) | 3 | 4.8 | 419 | 140 |
| 1.2 | 4,4′-bipyridine (L2) | 3 | 4.2 | 411 | 137 |
| 1.3 | 4,4′-bipyridine (L2) | 5 | 5.4 | 489 | 99 |
| 1.4 | bathocuproin (L3) | 3 | 3.1 | 310 | 104 |
| 1.5 | phenanthroline (L4) | 3 | 2.1 | 155 | 52 |

### Example 2

### Reaction Using Various Glass Reactors with n-Octane as Substrate and the Additive L2 (4,4′-bipyridine)

$$\begin{matrix}n \text{-} octane \\ 30 mmol\end{matrix} {{\to }^{\begin{matrix}RhCl ( CO ) {( {PMe}_{3} )}_{2} ( 0.004 mmol ) + \\ {4.4}^{′} \text{-} bipyridine ( 0.02 mmol )\end{matrix}}}_{ hv ( I = 320 \text{-} 500 nm ) } octenes$$

Schlenk vessels with wall thicknesses of 1.2 mm, 1.6 mm, 1.8 mm and 3 mm, and also a jacketed three-neck photoreactor and an internally reflective jacketed three-neck photoreactor having a wall thickness of 2.3 mm, provided with a reflux condenser and magnetic stirrer, were each charged with 1.3 mg of Rh(PMe₃)₂(CO)Cl (0.004 mmol) and 3.1 mg of 4,4′-bipyridine (L2, 0.02 mmol). Subsequently, the respective reaction vessel was evacuated and filled with argon three times, in order to achieve inert conditions. Subsequently, 5 ml of n-octane (30 mmol) were added. An argon stream was applied in order to remove hydrogen formed. The reactors were covered with aluminium foil and the light source having a wavelength of 320-500 nm (Lumatec Superlite 400) was switched on. The mixture was stirred at 1000 min⁻¹ for seven or five hours. After the reaction, the light source was switched off and the reaction solution was cooled down. The yield was determined by gas chromatography with isooctane as internal standard.

Table 2 shows conditions, yields and conversion rates.

TABLE 2

| | Glass vessel | | | | |
|---|---|---|---|---|---|
| No.^{a} | (wall thickness, mm) | Time (h) | Yield (%) | TON | TOF (h⁻¹) |
| 2.1 | Schlenk vessel (1.2)^{b} | 7 | 19.7 | 1518 | 217 |
| 2.2 | Schlenk vessel (1.2) | 5 | 16.7 | 1353 | 270 |
| 2.3 | Schlenk vessel (1.6) | 5 | 14.8 | 1268 | 253 |
| 2.4 | Schlenk vessel (1.8) | 5 | 12.3 | 1069 | 211 |
| 2.5 | Schlenk vessel (3.0) | 5 | 9.2 | 746 | 150 |
| 2.6 | Photoreactor (2.3) | 5 | 5.2 | 489 | 99 |
| 2.7 | Internally reflective | 5 | 8.0 | 636 | 127 |
| | photoreactor (2.3) | | | | |

^{a}In the photoreactor: light, glass, argon, solution; in the Schlenk vessel: light, glass, solution.
^{b}Composition: 7% 1-octene, 9% 2-octene, 1% 3-octene, 3% 4-octene and traces of dienes.

### Example 3

### Reaction Using Various Glass Reactors with Cycloctane as Substrate and the Additive L2 (4,4′-bipyridine)

$$\begin{matrix}cyclocotane \\ 30 mmol\end{matrix} {{\to }^{\begin{matrix}RhCl ( CO ) {( {PMe}_{3} )}_{2} ( 0.004 mmol ) + \\ {4.4}^{′} \text{-} bipyridine ( 0.02 mmol )\end{matrix}}}_{ hv ( λ = 320 \text{-} 500 nm ) } cyclooctene$$

The reaction was effected analogously to Example 2 using 4 ml of cyclooctane (30 mmol). Table 3 shows conditions, yields and conversion rates.

TABLE 3

| | Glass vessel (wall | | | | |
|---|---|---|---|---|---|
| Entry^{a} | thickness, mm) | Time (h) | Yield (%) | TON | TOF (h⁻¹) |
| 3.1 | Schlenk vessel (1.2) | 7 | 40 | 2883 | 411 |
| 3.2 | Schlenk vessel (1.2) | 5 | 38.4 | 2747 | 549 |
| 3.3 | Schlenk vessel (1.6) | 5 | 23.8 | 1741 | 348 |
| 3.4 | Schlenk vessel (1.8) | 5 | 23.8 | 1741 | 348 |
| 3.5 | Schlenk vessel (3.0) | 5 | 8 | 618 | 123 |
| 3.6 | Photoreactor (2.3) | 5 | 5.2 | 460 | 92 |

^{a}In the photoreactor: light, glass, argon, solution; in the Schlenk vessel: light, glass, solution. Small traces of an unidentified product with m/z = 220 (via GC-MS) and <1% for Entry 1.

### Example 4

In analogy to Examples 2 and 3, further substrates (30 mmol of each) were used, with use of a 1.2 mm-thick single-wall Schlenk vessel.

$$\begin{matrix}alkane \\ 30 mmol\end{matrix} {{\to }^{\begin{matrix}RhCl ( CO ) {( {PMe}_{3} )}_{2} ( 0.004 mmol ) + \\ {4.4}^{′} \text{-} bipyridine ( 0.02 mmol )\end{matrix}}}_{ \begin{matrix}hv ( I = 320 \text{-} 500 nm ) \\ 1.2 mm Schlenk vessel\end{matrix} } alkenes$$

Table 4 shows substrates, conditions, yields and conversion rates.

TABLE 4

| Entry | Alkane | Time (h) | Yield (%) | TON | TOF (h⁻¹) |
|---|---|---|---|---|---|
| 4.1^{a} | Cyclohexane | 7 | 13 | 975 | 195 |
| 4.2 | | 5 | 12.1 | 951 | 191 |
| 4.3^{b} | Methylcyclohexane | 7 | 16.5 | 1200 | 171 |
| 4.4 | | 5 | 16.1 | 1150 | 171 |
| 4.5^{c} | n-Hexane | 7 | 6.5 | 462 | 66 |
| 4.6^{c,d} | | 5 | 5.6 | 414 | 83 |
| 4.7^{e} | 2-Methylpentane | 7 | 4.7 | 368 | 53 |
| 4.8^{e,f} | | 5 | 4.3 | 342 | 68 |
| 4.9 | n-Dodecane | 7 | 14.6 | 1100 | 220 |
| 4.10^{g} | Tetralin | 7 | 5 | 357 | 51 |
| 4.11 | Decalin (cis + trans) | 7 | 2.2 | 165 | 24 |
| 4.12 | Indoline | 7 | 9.3 | 708 | 101 |

^{a}Traces of an unknown product with m/z = 164 (via GC-MS).
^{b}Composition: 0.8% methylenecyclohexane, 1.6% 1-methyl-1-cyclohexene, 10% 1-methyl-3-cyclohexene, 4% 1-methyl-4-cyclohexene and traces of cyclopentene.
^{c}Alkane loss.
^{d}Composition: 0.3% 1-hexene, 4.8% 2-hexene, 1.4% 3-hexene.
^{e}Significant alkane loss.
^{f}Composition: 3.4% 4-methyl-1-pentene, 1% 2-methyl-1-pentene, 0.3% 4-methyl-2-pentene & 2-methyl-2-pentene.
^{g}4% 1,2-dihydronaphthalene, ~1% 1,4-dihydronaphthalene and traces of naphthalene.

### Example 5

### Simultaneous Conversion of Linear and Cyclic Alkanes

$$\begin{matrix}n \text{-} octane \\ 15 mmol\end{matrix} + \begin{matrix}cyclooctane \\ 15 mmol\end{matrix} {{\to }^{\begin{matrix}RhCl ( CO ) {( {PMe}_{3} )}_{2} ( 0.004 mmol ) + \\ {4.4}^{′} \text{-} bipyridine ( 0.02 mmol )\end{matrix}}}_{\begin{matrix}hv ( 320 \text{-} 500 nm ) , 7 h \\ 1.2 mm Schlenk vessel\end{matrix}} \begin{matrix}octenes + cyclooctene \\ ( 1 \text{:} 6 )\end{matrix}$$

A Schlenk vessel having a wall thickness of 1.2 mm, provided with a reflux condenser and magnetic stirrer, was charged with 1.3 mg of Rh(PMe₃)₂(CO)Cl (0,004 mmol) and 3.1 mg of 4,4′-bipyridine (L2, 0.02 mmol). Subsequently, the reaction vessel was evacuated and filled with argon three times, in order to achieve inert conditions. Then 2.5 ml of n-octane (15 mmol) and 2 ml of cyclooctane (15 mmol) were added. An argon stream was applied in order to remove hydrogen formed. The reactor was covered with aluminium foil and the light source having a wavelength of 320-500 nm (Lumatec Superlite 400) was switched on. The mixture was stirred at 1000 min⁻¹ for seven hours. After the reaction, the light source was switched off and the reaction solution was cooled down. The yield was determined by gas chromatography with isooctane as internal standard, and is shown in Table 5.

TABLE 5

| Entry | Alkene | Yield (%) | TON | TOF (h⁻¹) |
|---|---|---|---|---|
| 5.1 | Octene | 3.0 | 200 | 29 |
| | Cyclooctene | 35 | 1193 | 170 |

By way of summary for the alkane dehydrogenation, it can be stated that the yield or reactivity of the substrates is determined principally by the enthalpy of the dehydrogenation. The higher yield in the cyclooctane dehydrogenation (Example 3.2) can accordingly be explained by the lower enthalpy of 23.3 kcal/mol, while a lower yield is recorded for cyclohexane, having a significantly higher enthalpy of 28.2 kcal/mol (Example 4.2). The enthalpy for methylcyclohexane is precisely in between them at 26.5 kcal/mol, as is the yield of the corresponding dehydrogenation product (Example 4.4). The enthalpy for linear C6-C10 alkanes is between 27 and 30 kcal/mol. Therefore, the endothermicity is the crucial factor in the examples presented. This is also verified by the above control experiment in which n-octane and cyclooctane were used in a 1:1 mixture. Table 5 convincingly shows the preference for the dehydrogenation of the cyclooctane.

### Example 6

### Dehydrogenation of Alcohols

$$\begin{matrix}alcohol \\ 30 mmol\end{matrix} {{\to }^{\begin{matrix}RhCl ( CO ) {( {PMe}_{3} )}_{2} ( 0.004 mmol ) + \\ {4.4}^{′} \text{-} bipyridine ( 0.02 mmol )\end{matrix}}}_{\begin{matrix}hv ( 320 \text{-} 500 nm ) , 3 \text{-} 5 h \\ 1.2 mm Schlenk vessel\end{matrix}} product$$

A Schlenk vessel having a wall thickness of 1.2 mm, provided with a reflux condenser and magnetic stirrer, was charged with 1.3 mg of Rh(PMe₃)₂(CO)Cl (0,004 mmol) and 3.1 mg of 4,4′-bipyridine (L2, 0.02 mmol). Subsequently, the reaction vessel was evacuated and filled with argon three times, in order to achieve inert conditions. Then 3.2 ml of cyclohexanol, 3.9 ml of cyclooctanol, 2.3 ml of isopropanol, 1-hexanol, 1-nonanol (30 mmol of each) were added. An argon stream was applied in order to remove hydrogen formed. The reactor was covered with aluminium foil and the light source having a wavelength of 320-500 nm (Lumatec Superlite 400) was switched on. The mixture was stirred at 1000 min⁻¹ for 3 hours. After the reaction, the light source was switched off and the reaction solution was cooled down. The yield was determined by gas chromatography according to the above reaction equation.

Table 6 shows alcohol substrates used, conditions, yields and conversion rates.

TABLE 6

| | | | Time | Yield | | TOF |
|---|---|---|---|---|---|---|
| Entry | Substrate | Product | (h) | (%) | TON | (h⁻¹) |
| 7.1 | Cyclohexanol | Cyclohexanone | 3 | 3.9 | 288 | 96 |
| 7.2 | Cyclooctanol | Cyclooctanone | 3 | 6.8 | 454 | 151 |
| 7.3 | Isopropanol | Acetone | 3 | 2.5 | 189 | 38 |
| 7.4 | 1-Hexanol | Hexanal | 3 | 0.5 | 38 | 13 |
| 7.5 | 1-Nonanol | Nonanal | 3 | 1.3 | 101 | 34 |

### Example 7

### Reaction Using a 1.2 mm-Thick Single-Wall Schlenk Vessel with n-Octane as Substrate and the Additive L2 (4,4′-bipyridine) in the Presence of CO₂

$$\begin{matrix}n \text{-} octane \\ 30 mmol\end{matrix} {{\to }^{\begin{matrix}RhCl ( CO ) {( {PMe}_{3} )}_{2} ( 0.004 mmol ) + \\ {4.4}^{′} \text{-} bipyridine ( 0.02 mmol )\end{matrix}}}_{ hv ( λ = 320 \text{-} 500 nm ) } octenes$$

A Schlenk vessel having a wall thickness of 1.2 mm, provided with a reflux condenser and magnetic stirrer, was charged under argon with 1.3 mg of Rh(PMe₃)₂(CO)Cl (0.004 mmol) and 3.1 mg of 4,4′-bipyridine (L2, 0.02 mmol). Subsequently, the reaction vessel was evacuated and filled with argon three times, in order to achieve inert conditions. Subsequently, 5 ml of n-octane (30 mmol) were added. The stirrer speed was set to 1000 min⁻¹ and the glass vessel was covered with aluminium foil. A metal capillary was used to pass a CO₂ stream through the solution. Then the Lumatec Superlite 400 light source used was switched on. After the reaction, the light source was switched off, the CO₂ stream was stopped, the reaction solution was cooled down and the yield was determined by gas chromatography with isooctane as internal standard.

TABLE 7

| | | |
|---|---|---|
| Time | without CO₂ | with CO₂ |

| Entry | (h) | Yield (%) | TON | Yield (%) | TON |
|---|---|---|---|---|---|
| 1 | 3 | 11.3 | 850 | 12.3 | 881 |
| 2 | 5 | 16.7 | 1353 | 16.0 | 1197 |
| 3 | 7 | 19.7 | 1518 | 17.0 | 1250 |
| 4 | 14 | 19.6 | 1495 | 23.0 | 1666 |

### Example 8

### Reaction Using a 1.2 mm-Thick Single-Wall Schlenk Vessel with Cyclooctane as Substrate, the Additive L2 (4,4′-bipyridine) and [Rh(CO)Cl(PPh₂CH₂PPh₂)]₂ as Catalyst

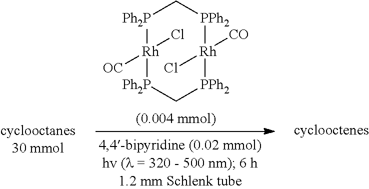

A Schlenk vessel having a wall thickness of 1.2 mm, provided with a reflux condenser and magnetic stirrer, was charged under argon with 4.4 mg of [Rh(CO)Cl(PPh₂CH₂PPh₂)]₂ (0.004 mmol) and 3.1 mg of 4,4′-bipyridine (L2, 0.02 mmol). Subsequently, the reaction vessel was evacuated and filled with argon three times, in order to achieve inert conditions. Then 4 ml of cyclooctane (30 mmol) were added. An argon stream was applied in order to remove hydrogen formed. The reactor was covered with aluminium foil and the light source having a wavelength of 320-500 nm (Lumatec Superlite 400) was switched on. After the reaction, the light source was switched off, the argon stream was shut down, the reaction solution was cooled down and the yield was determined by gas chromatography with isooctane as internal standard.

TABLE 8

| Entry | Time (h) | Yield (%) | TON | TOF (h⁻¹) |
|---|---|---|---|---|
| 1 | 6 | 9 | 650 | 108 |

1. A process for dehydrogenation of an alkane or an alcohol, comprising:
irradiating the alkane or alcohol in a reaction mixture comprising a rhodium complex and a Lewis base to remove hydrogen from the alkane or alcohol;
wherein
a hydrogen acceptor is not present, and
the rhodium complex comprises a ligand of an organic phosphorus(III) compound.

2. The process according to claim 1,
wherein the Lewis base is an organic amine.

3. The process according to claim 1,
wherein the Lewis base is a heterocyclic amine.

4. The process according to claim 1,
wherein the Lewis base is at least one selected from the group consisting of a bipyridine, bathocuproin and phenanthroline.

5. The process according to claim 4,
wherein the Lewis base is a bipyridine and is 2,2-bipyridine and/or 4,4-bipyridine.

6. The process according to claim 1,
wherein the Lewis base used is bathocuproin or phenanthroline.

7. The process according to claim 1,
wherein the reaction is conducted in the presence of CO₂.

8. The process according to claim 1,
wherein the irradiation is conducted with light of wavelength 320 nm to 500 nm.

9. The process according to claim 1,
wherein a temperature of the dehydrogenation is from 45° C. to 120° C.

10. The process according to claim 1,
wherein the rhodium complex is of formula (I):
Rh(L¹)₂(CO)X (I)
wherein
L¹ is P(C₁-C₅alkyl)₃, and
X is chloride, bromide or acetate.

11. The process according to claim 10, wherein
L¹ is PMe₃ or PtBu₃.

12. The process according to claim 10, wherein X is chloride.

13. The process according to claim 1, wherein the rhodium complex is of formula (II):
Rh₂(L²)₂(CO)₂(X)₂ (II)
wherein
L² is P(Ph)₂(CH₂)ₙ P(Ph)₂,
n is from 1 to 3, and
X is chloride, bromide or acetate.

14. The process according to claim 13, wherein
L² is P(Ph)₂(CH₂) P(Ph)₂.

15. The process according to claim 13, wherein X is chloride.

16. The process according to claim 1, wherein
a carbon chain length of the alkane or the alcohol is from 2 to 30 carbon atoms.

17. The process according to claim 1, further comprising removing the hydrogen from the reaction mixture.

18. The process according to claim 1, wherein the dehydrogenation is conducted in a glass reactor or a metal reactor comprising glass.

19. The process according to claim 1, wherein the dehydrogenation is conducted in a single-wall glass reactor having a wall thickness of from 1.0 to 3.0 mm.

20. The process according to claim 1, wherein a turnover number is greater than 1000.
